(19) United States
(12) Patent Application Publication
(10) Pub. No.: US 2025/0235116 A1
(43) Pub. Date: Jul. 24, 2025

(54) FLEXIBLE ULTRASONIC TRANSDUCER CAPABLE OF DETECTING SKIN IMPEDANCE, DRIVING DEVICE AND CONTROL METHOD

(71) Applicant: NANJING KHONS MEDTECH CO., LTD., Nanjing (CN)

(72) Inventors: Xiangqing KONG, Nanjing (CN); Enguang ZHANG, Nanjing (CN); Ruibiao GUO, Nanjing (CN); Jing CAI, Nanjing (CN); Feifei CHE, Nanjing (CN)

(73) Assignee: NANJING KHONS MEDTECH CO., LTD., Nanjing (CN)

(21) Appl. No.: 18/947,286

(22) Filed: Nov. 14, 2024

(30) Foreign Application Priority Data
Jan. 23, 2024 (CN) 202410093545.5

(51) Int. Cl. A61B 5/0531; A61B 8/00

(52) U.S. Cl. CPC A61B 5/0531; A61B 8/4488; A61B 8/4494

(57) ABSTRACT

A flexible ultrasonic transducer capable of detecting skin impedance, driving device and control method, the ultrasonic transducer includes coupling, electrode, array element and backing layers, and coupling layer includes a non-conductive and conductive flexible material; electrode layer is located below coupling layer, and second metal layer for connecting conductive flexible material and driving device, and fourth metal layer for connecting ground are arranged in electrode layer; array element layer is fit together with electrode layer and includes N transducer array elements, array element drive wires and flexible material, and flexible material fills among transducer array elements; backing layer is located below array element layer. The flexible transducer in conjunction with the driving device and control method can realize detection of skin impedance. Based on skin impedance, fit degree of transducer can be detected, and real-time evaluation and feedback control of ultrasonic emission effect can be realized in emission process.

### TECHNICAL FIELD

The present invention relates to a flexible ultrasonic transducer capable of detecting skin impedance, a driving device and a control method and pertains to the technical field of flexible ultrasound.

### BACKGROUND ART

At present, the ultrasonic devices used in the field of dermatology, such as ultrasonic physiotherapy instruments, ultrasonic therapeutic instruments and ultrasonic medicine guide instruments, typically use piezoelectric ceramic pieces as core components of ultrasonic transducers (ultrasonic probes). These transducers typically comprise piezoelectric ceramic pieces, electrode connecting lines and housing structures, and may also include temperature detection circuits, humidity detection circuits, etc. Although these ultrasonic probes are excellent in generating ultrasonic waves and can also detect the operating condition of the transducers, they have a limitation, that is, they cannot timely detect the changes of the tissue after ultrasonic irradiation, so they cannot perform feedback control of ultrasonic emission energy.

Further, these transducers typically have rigid surfaces, so when they are applied to uneven skin, it is difficult to achieve a desirable coupling with the skin, and in actual use, it is necessary to apply a coupling agent between the transducer surface and the skin surface, causing much inconvenience in use.

### SUMMARY OF THE INVENTION

An objective of the present invention is to provide a flexible ultrasonic transducer capable of detecting skin impedance, a driving device and a control method to detect skin impedance in the process of ultrasonic emission, thereby timely detecting the changes of the tissue after ultrasonic irradiation and performing feedback control of ultrasonic emission according to the detection results. Further, the transducer is designed to be made of a flexible material as a whole, can be well conformal to changes and can perfectly solve the problem of coupling between the transducer and the skin without a coupling agent.

In order to achieve the foregoing objective, the present invention adopts the following technical scheme:

A flexible ultrasonic transducer capable of detecting skin impedance, comprising a coupling layer, an electrode layer, an array element layer and a backing layer, wherein:

The coupling layer comprises a non-conductive flexible material and a conductive flexible material;

The electrode layer is located below the coupling layer, and a second metal layer for connecting the conductive flexible material and a driving device, and a fourth metal layer for connecting the ground are arranged in the electrode layer;

The array element layer is fit together with the electrode layer and comprises N transducer array elements, array element drive wires and a flexible material, and the flexible material fills among the transducer array elements;

The backing layer is located below the array element layer.

Further, the coupling layer is an odd number of times as thick as a quarter of the ultrasonic wavelength.

Further, an orifice is opened in the non-conductive flexible material and filled with the conductive flexible material.

Further, the direct current impedance and alternating current impedance of the non-conductive flexible material are greater than the measured skin impedance; the direct current impedance and alternating current impedance of the conductive flexible material are smaller than the measured skin impedance.

Still further, cotton slots are arranged on the coupling layer and intended to contain medicine guide cotton.

Further, the electrode layer comprises a first insulating layer, a second metal layer, a third insulating layer, a fourth metal layer and a fifth insulating layer arranged in a stacking manner in turn.

Still further, the second metal layer is provided with a skin impedance detection electrode array, the skin impedance detection electrode array comprises N metal conductors, and two ends of each metal conductor are provided with a first metal electrode and a second metal electrode, respectively.

Still further, the metal conductors adopt a serpentine structure that curves back and forth.

Still further, the first metal electrode is electrically connected to the conductive flexible material, and the second metal electrode is electrically connected to the driving device.

Still further, an orifice is arranged in the first insulating layer above the first metal electrode and filled with the conductive flexible material, and the first metal electrode is electrically connected to the conductive flexible material.

Still further, the fourth metal layer L24 is electrically connected to the ground.

Further, the array element layer comprises N transducer array elements, array element drive wires and a flexible material, and the flexible material fills among the transducer array elements, and the array element drive wires are arranged below the transducer array elements and the flexible material and electrically connected to the transducer array elements.

Still further, the minimum distance Dₘᵢₙ between the transducer array elements is calculated according to the following formula:

$${D}_{min} = 2 h sin \frac{π - β}{2}$$

Where, h is the thickness of the transducer array elements, and β is a conformal skin angle required for the ultrasonic transducer.

Still further, top electrodes of the transducer array elements extend through the side faces of the array elements to the bottom surfaces of the array elements and are connected to the array element drive wires, and bottom electrodes of the transducer array elements are connected to the array element drive wires.

Still further, the transducer array elements are planar array elements or curved array elements, the convex sides of the curved array elements face upward, the thickness of the non-conductive flexible material above the curved array elements is greater than D_{L03}, and the calculation formula for D_{L03} is as follows:

$${D}_{L 03} = \frac{D + d}{2 tan α} - r - h$$

where, α is the radiation angle of the curved array elements, r is the curvature radius of the curved array elements, d is the width of the curved array elements, h is the thickness of the curved array elements, and D is the actual distance between array elements.

A driving device used in the flexible ultrasonic transducer and comprising a control unit, an impedance detection unit and an array element driving unit, wherein the control unit is electrically connected to the impedance detection unit and the array element driving unit, and the array element driving unit is electrically connected to the transducer array elements, and applies electric driving signals to the transducer array elements, respectively, thereby achieving ultrasonic emission.

Further, the impedance detection unit contains N impedance detection circuits, the N impedance detection circuits are electrically connected to the metal conductors of the skin impedance detection electrode array and detection resistors R are connected at signal return ends of the metal conductors; the impedance detection unit controls each impedance detection circuit in turn to apply an electrical excitation signal u₀ to the metal conductors, and at the same time, detects the return electrical signal uₙ of each channel after passing through the skin tissue, where n is the channel number.

Still further, the electric excitation signal u₀ is an alternating current signal, the control unit extracts one or more periodic signals u₀′ after the electric excitation signal u₀ passes the zero point for the first time, and one or more periodic signals uₙ′ after the return electrical signal uₙ passes the zero point for the first time, and the periodic signals u₀′ and uₙ′ have the same number of periods.

Still further, the control unit calculates the complete impedance Z_{total,n} of the entire signal link for each channel according to the following formula:

$${Z}_{total , n} = \frac{{u}_{0}^{′} R}{{u}_{n}^{′}} - R$$

where, u₀′ is one or more periodic signals after the electric excitation signal u₀ passes the zero point for the first time, uₙ′ is one or more periodic signals after the return electrical signal uₙ passes the zero point for the first time, and R is the resistance of the detection resistor;

The control unit calculates the impedance Zₙ of the skin tissue according to the following formula:

$${Z}_{n} = {Z}_{total , n} - 2 {Z}_{t , n} - 2 {Z}_{m , n} - 2 {Z}_{l , n} - {Z}_{s , n}$$

Where, n is a channel number, Z_{t,n} is the equivalent impedance of the detection electrode L21, Z_{m,n} is the equivalent impedance of the conductive flexible material, Z_{l,n} is the equivalent impedance of the metal conductors, and Z_{s,n} is the equivalent impedance of the signal link on the impedance detection unit.

Still further, the electric excitation signal u₀ is a direct current signal, and the control unit calculates the complete impedance Z_{total,n} of the entire signal link for each channel according to the following formula:

$${Z}_{total , n} = \frac{{u}_{0} R}{{u}_{n}} - R$$

Where, u₀ is the electric excitation signal, uₙ is the return electrical signal, and R is the resistance of a detection resistor;

The control unit calculates the impedance Zₙ of the skin tissue according to the following formula:

$${Z}_{n} = {Z}_{t o tal , n} - 2 {Z}_{t , n} - 2 {Z}_{m , n} - 2 {Z}_{l , n} - {Z}_{s , n}$$

Where, n is a channel number, Z_{t,n} is the equivalent impedance of the detection electrode L21, Z_{m,n} is the equivalent impedance of the conductive flexible material, Z_{l,n} is the equivalent impedance of the metal conductors, and Z_{s,n} is the equivalent impedance of the signal link on the Z impedance detection unit.

A control method of the driving device of the flexible ultrasonic transducer, comprising the following steps:
S01: controlling the impedance detection unit to apply an electric excitation signal u₀ to each channel impedance detection electrode in turn, wherein the electric excitation signal u₀ is an alternating current signal or a direct current signal, if the electric excitation signal u₀ is an alternating current signal, then going to S02 and S03, and if the electric excitation signal u₀ is a direct current signal, then going to S02 and S04;
S02: controlling the impedance detection unit to detect a return electrical signal uₙ of each channel impedance detection electrode after passing through the skin tissue;
S03: extracting three periodic signals u₀′ after the electric excitation signal u₀ passes the zero point for the first time, and three periodic signals uₙ′ after the return electrical signal uₙ passes the zero point for the first time if the electric excitation signal u₀ is an alternating current signal; at the same time, calculating the complete impedance Z_{total,n} of the entire signal link for each channel according to the following formula:

$${Z}_{total , n} = \frac{{u}_{0}^{′} R}{{u}_{n}^{′}} - R ;$$

where, u₀′ is one or more periodic signals after the electric excitation signal u₀ passes the zero point for the first time, uₙ′ is one or more periodic signals after the return electrical signal uₙ passes the zero point for the first time, and R is the resistance of a detection resistor;
S04: calculating the complete impedance Z_{total,n} of the entire signal link for each channel according to the following formula by the control unit if the electric excitation signal u₀ is a direct current signal:

$${Z}_{total , n} = \frac{{u}_{0} R}{{u}_{n}} - R ;$$

where, u₀ is an electric excitation signal, uₙ is a return electrical signal, and R is the resistance of a detection resistor;
S05: calculating the impedance Zₙ of the skin tissue according to the following formula:

$${Z}_{n} = {Z}_{total , n} - 2 {Z}_{t , n} - 2 {Z}_{m , n} - 2 {Z}_{l , n} - {Z}_{s , n}$$

Where, n is a channel number, Z_{t,n} is the equivalent impedance of the detection electrode L21, Z_{m,n} is the equivalent impedance of the conductive flexible material, Z_{l,n} is the equivalent impedance of the metal conductors, and Z_{s,n} is the equivalent impedance of the signal link on the impedance detection unit;
S06: calculating the deviation Zₛₙ of each value Zₙ;

$${Z}_{s n} = \frac{{Z}_{n} - μ}{\sqrt{\frac{{\sum }_{n = 1}^{N} {( {Z}_{n} - μ )}^{2}}{N}}}$$

Where, μ is a mean value of all values Zₙ;
S07: comparing Zₛₙ with a preset deviation threshold, and marking channels that exceed the deviation threshold as abnormal channels;
S08: applying an electrical signal to non-abnormal channels by means of the array element driving unit, thereby achieving ultrasonic emission;
S09: during ultrasonic emission, detecting the skin impedance values of N channels in turn by means of the impedance detection unit again, and marking them as Zₙ′;
S10: calculating the change in skin impedance of each channel after ultrasonic irradiation according to the following formula and marking it as Δkₙ:

$$Δ {k}_{n} = \vert 1 - \frac{{Z}_{n}^{′} - {Z}_{n}}{{Z}_{n}} \vert$$

S11: comparing Δkₙ with a preset dose threshold, stopping emitting ultrasonic waves to channels of which Δkₙ is greater than the dose threshold, and continuing to emit ultrasonic Ak, waves to other channels; and

S12: repeating S03 to S07 until all channels reach the dose threshold. Compared with the prior art, the present invention has the following advantages:

(1) In the present application, the clearances between the coupling layer and the array elements of the flexible transducer are filled with a flexible material, and the metal conductors adopt the design of spring wires or serpentine wires, so they are flexible and ductile and can be well conformal to and fit with uneven skin.

(2) In the present application, the flexible transducer is designed with electrodes that can be used to detect skin impedance, and can realize the detection of skin impedance in conjunction with the driving device and the control method. Based on the skin impedance, the fit degree of the transducer can be detected and real-time evaluation and feedback control of the ultrasonic emission effect can be realized in the emission process.

(3) In the present application, the flexible transducer can independently control the ultrasonic emission of each array element according to the skin impedance of each channel, and compared with an ultrasonic transducer controlling a single array element and a plurality of array elements in a unified way, it can better ensure the ultrasonic irradiation effects at different locations and can effectively solve the problem of inconsistent ultrasonic irradiation effects caused by differences in fit degree, skin tissue density and tissue ultrasonic sensitivity at different locations.

### BRIEF DESCRIPTION OF THE DRAWINGS

FIG. 1 is a schematic view of an embodiment of a flexible transducer with a 2×4 array of transducer array elements distributed rectangularly;

FIG. 2 is a schematic view of a coupling layer L1 in the embodiment shown in FIG. 1;

FIG. 3 is a schematic view of an alternative implementation manner for the coupling layer L1 in the embodiment shown in FIG. 1;

FIG. 4 is a schematic view of an electrode layer L2 in the embodiment shown in FIG. 1;

FIG. 5 is a schematic side view of the embodiment shown in FIG. 1;

FIG. 6 is a schematic side view of an embodiment of a curved array element;

FIG. 7 is a schematic top view of an array element layer L3 in the embodiment shown in FIG. 1;

FIG. 8 is a schematic bottom view of the array element layer L3 in the embodiment shown in FIG. 1. The angle of view is opposite to that of FIG. 5;

FIG. 9 is a schematic view of calculation of the minimum distance of the array elements;

FIG. 10 is a schematic view of calculation of the thickness of the flexible material L12 in an embodiment of the curved array elements;

FIG. 11 is a schematic view of an array element electrode;

FIG. 12 is a schematic view of a driving device;

FIG. 13 is a schematic view of detection of skin impedance by an impedance detection unit;

FIG. 14 is an embodiment of an impedance detection circuit.

Description of reference signs: L1—coupling layer; L2—electrode layer; L3—array element layer; L4—backing layer; L11—conductive flexible material; L12—non-conductive flexible material; L13—cotton slot; L21—first insulating layer; L22—second metal layer; L23—third insulating layer; L24—fourth metal layer; L25—fifth insulating layer; L221—metal conductor; L222—first metal electrode; L223—second metal electrode; L31—transducer array element; L32—array element drive wire; L33—flexible material; L311—top electrode; L312—bottom electrode; L51—control unit; L52—impedance detection unit; L53—array element driving unit.

### DETAILED DESCRIPTION

The present invention will be further described in detail below in conjunction with the accompanying drawings and specific embodiments.

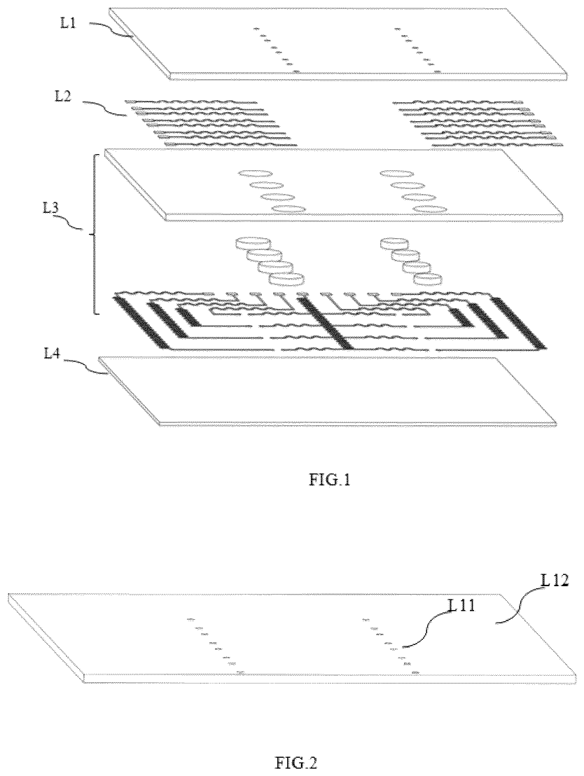

As shown in FIG. 1, the ultrasonic transducer in this embodiment comprises a coupling layer L1, an electrode layer L2, an array element layer L3 and a backing layer L4.

As shown in FIG. 2, the coupling layer L1 comprises a non-conductive flexible material L12 and a conductive flexible material L11. In conjunction with FIG. 5, the conductive flexible material L11 fills an orifice above a metal electrode L222, and in this embodiment, the orifice is circular with a diameter of 0.6 mm, in the same size as the metal electrode L222.

For a better coupling with skin, the conductive flexible material L11 and the non-conductive flexible material L12 are equally high.

Further, the non-conductive flexible material L12 is silicone rubber. In order to meet the requirements of impedance measurement, the direct current impedance and alternating current impedance of the non-conductive flexible material L12 are greater than the measured skin impedance, recommended to be greater than 10 times of the measured skin impedance in actual implementation. In this embodiment, the measured skin impedance is 50˜ 500 kΩ and the impedance of the non-conductive flexible material shall be greater than 5 MΩ, so in this embodiment, silicone rubber is selected as the non-conductive flexible material.

Further, the conductive flexible material L11 is conductive hydrogel filled with conductive fibers. In actual implementation, the direct current impedance and alternating current impedance of the conductive flexible material L11 need to be smaller than the measured skin impedance, and the smaller, the better. In this embodiment, conductive hydrogel filled with conductive fibers is selected, of which direct current impedance and alternating current impedance are approximately 1 kΩ, ⅕ of the minimum measured skin impedance, and is an ideal choice.

Further, the thickness of the coupling layer L1 is calculated according to the operating frequency of the transducer array elements:

In this embodiment, according to the operating frequency f=1 MHz of the transducer array elements, the ultrasonic wavelength λ is calculated to be 1.5 mm, and the thickness of the coupling layer L1 is designed to be

$$\frac{5}{4} λ ,$$

i.e., 1.875 mm.

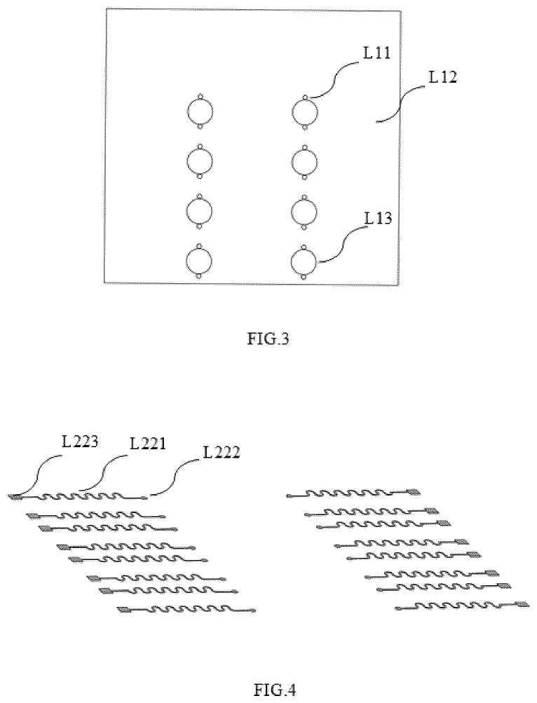

In another embodiment, as shown in FIG. 3, the coupling layer L1 is further provided with cotton slots L13 above the transducer array elements L31, and the cotton slots L13 are in a diameter same as the size of the transducer array elements, i.e., 8 mm, and are intended to contain medicine guide cotton.

Further, as shown in FIG. 1 and FIG. 4, the electrode layer L2 and the coupling layer L1 are fit together. At the same time, in conjunction with FIG. 5, the electrode layer L2 avoids the ultrasonic irradiation range of the transducer array elements L31. After the flexible ultrasonic transducer is assembled, the upper surfaces of the transducer array elements L31 are flush with or lower than the upper surface of the electrode layer L2 so as to effectively avoid the interference of ultrasonic irradiation to the electrode layer L2.

Further, the electrode layer L2 contains N skin impedance detection electrodes. In order to make the whole transducer flexible, the N skin impedance detection electrodes are designed in the form of independent spring wires or serpentine wires.

Further, as shown in FIG. 5, each skin impedance detection electrode comprises a first insulating layer L21, a second metal layer L22, a third insulating layer L23, a fourth metal layer L24 and a fifth insulating layer L25 arranged in a stacking manner in turn.

The second metal layer L22 comprises a first metal electrode L222, a metal conductor L221 and a second metal electrode L223. The first metal electrode L222, the metal conductor L221 and the second metal electrode L223 are electrically connected to each other, and the second metal electrode L223 is electrically connected to a driving device.

Further, as shown in FIG. 5, an orifice is arranged in the first insulating layer L21 above the metal electrode L222, which is filled with a conductive flexible material L11, so that the first metal electrode L222 is electrically connected to the conductive flexible material L11.

Further, considering the limitation of the distance between the transducer array elements L31, and the requirements for the machinability of the metal electrode L222 and the metal conductor L221 and for avoidance of the radiation sound field of the ultrasonic transducer array elements L31, the first metal electrode L222 is designed to be a circular electrode with a diameter of 0.6 mm, and the metal conductor L221 is designed in the form of a 1.0 mm wide serpentine wire. Further, the first metal electrode L222 is distributed in the periphery of the transducer array elements L31, thereby avoiding the radiation range of the sound field of the transducer array elements L31. In order to measure skin impedance in a better way, it is suggested to keep the distance between each pair of electrodes as large as possible in actual implementation. If the distance is too small, the measured skin impedance may be too small, the error is large and it is liable to noise interference.

Further, the fourth metal layer L24 is connected to the ground, thereby playing a role in controlling the impedance of the metal conductors L221 and isolating the interference of the transducer array element drive wires L32.

In this embodiment, the second metal layer L22 and the fourth metal layer L24 are made of copper.

To ensure that the electrode layer L2 is not broken during stretching, the third insulating layer L23 is made of a flexible material with certain toughness, and the material selected in this embodiment is polyimide (PI).

In this embodiment, the insulating material applied on the first insulating layer L21 and the fifth insulating layer L25 is green oil.

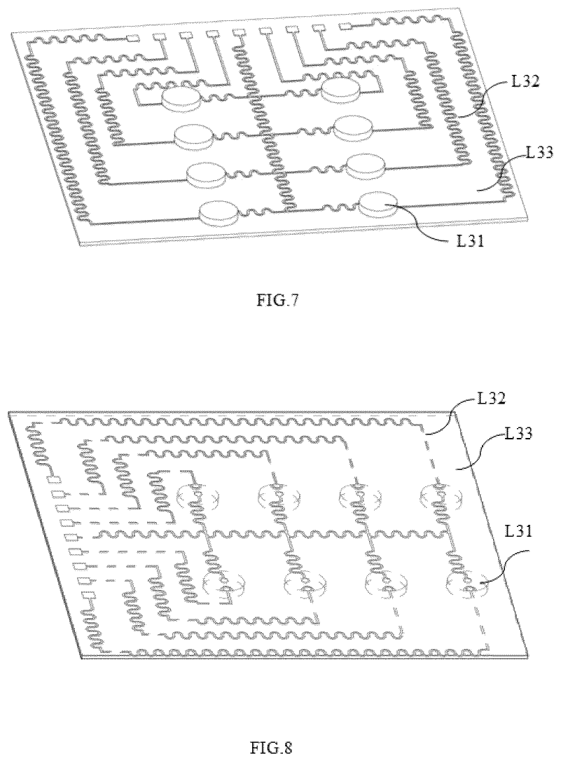

Further, as shown in FIG. 7, in conjunction with FIG. 1, the array element layer L3 is fit together with the electrode layer L2. In this embodiment, the array element layer contains 2×4, in total of 8, transducer array elements L31, as well as array element drive wires L32 and a flexible material L33, and the flexible material L33 is filled among the transducer array elements L31.

As shown in FIG. 8, in conjunction with FIG. 11, top electrodes L311 of the transducer array elements L31 extend through the side faces of the array elements to the bottom surfaces of the array elements and are connected to the array element drive wires L32. Bottom electrodes L312 of the transducer array elements are connected to the array element drive wires L32. In conjunction with FIG. 5 and FIG. 7, in this embodiment, the transducer array elements L31 are distributed in a rectangular 2×4 array, the transducer array elements L31 are planar array elements, with a diameter d of 8 mm, a thickness h of 1.63 mm and an operating frequency/of 1 MHz. It should be noted that in practical applications, the distribution form of the array of transducer array elements can be rectangular, circular, elliptical or in any other form, and the number of transducer array elements L31 can be any value.

Further, in conjunction of FIG. 9, in this embodiment, the conformal skin angle β required for the ultrasonic transducer is 110°, and the minimum value of the distance Dₘᵢₙ between the transducer array elements L31 calculated according to the following formula is 1.87 mm.

$${D}_{min} = 2 h sin \frac{π - β}{2}$$

Where, h is the thickness of the transducer array elements, and β is a conformal skin angle required for the ultrasonic transducer.

Further, in conjunction of FIG. 4, considering the requirements for the machinability of the metal electrode L222 and the metal conductor L221 and for avoidance of the radiation sound field of the ultrasonic transducer array element L31, the distance between the transducer array elements L31 is actually designed to be 3 mm.

In this embodiment, each of the transducer array elements can be controlled separately.

In this embodiment, the transducer array elements are planar array elements. As the effective sound field of the planar array elements is approximately quasi-straight, the maximum range of the axial vertical plane of the effective sound field is the area of the planar array elements. When the ultrasonic transducer is flat, the effective sound field of the ultrasonic transducer has clearances, and the skin or tissue in the clearance areas will not be subjected to effective ultrasonic irradiation, so this scheme is suitable for the case of a skin surface with a large curvature.

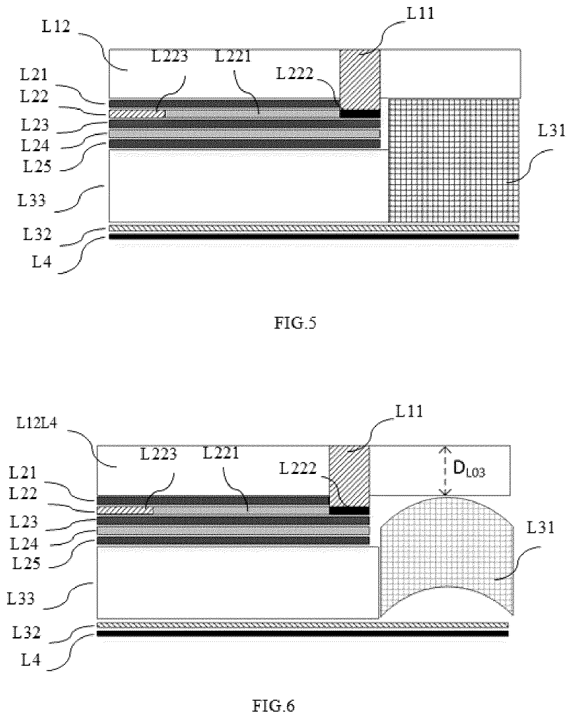

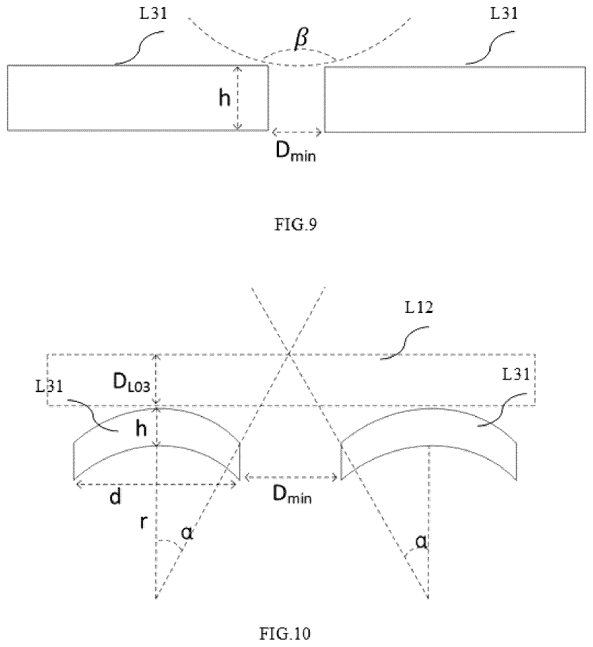

In another embodiment, as shown in FIG. 6 and FIG. 10, the transducer array elements can be changed into curved array elements, with a diameter d of 8 mm, a thickness h of 1.63 mm, a field angle α of 25°, and a curvature radius r of 8 mm. The convex surfaces of the curved array elements face upward. When the flexible material L12 above the curved array elements is thicker than D_{L03} and when the ultrasonic transducer is flat, the effective sound field of the ultrasonic transducer has no clearance, which can assure that all skin or tissue is subjected to effective ultrasonic irradiation, so this scheme is suitable for the case of smooth skin surface.

Further, D_{L03} is calculated according to the following formula to be 2.17 mm.

$${D}_{L 03} = \frac{D + d}{2 tan α} - r - h$$

where, α is the field angle of the curved array element, r is the curvature radius of the curved array element, d is the width of the curved array element, h is the thickness of the curved array element, and D is the actual distance between array elements.

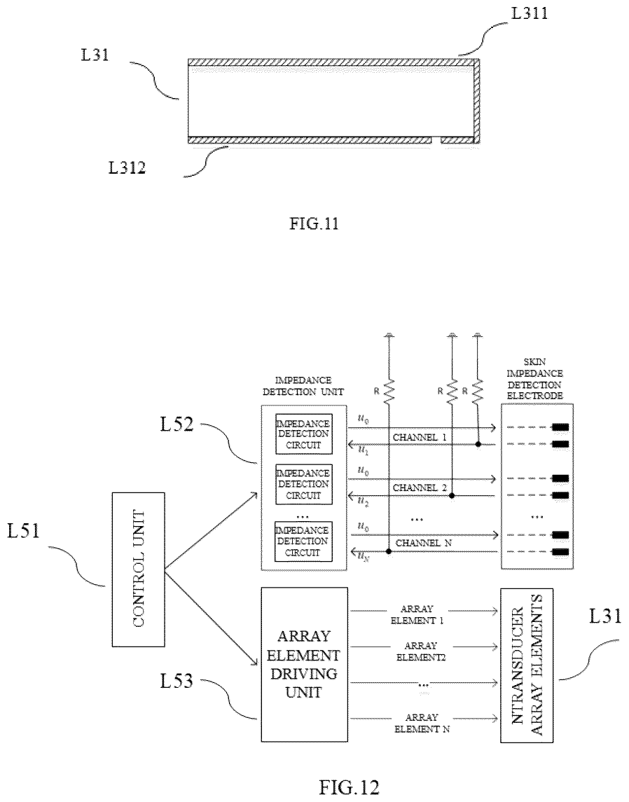

As shown in FIG. 12, in this embodiment, the driving device comprises a control unit L51, an impedance detection unit L52 and an array element driving unit L53. The control unit includes a storage medium, which stores an executable program executing the control method. The control unit L51 is electrically connected to the impedance detection unit L52 and the array element driving unit L53 and executes the control method. The array element driving unit L53 is electrically connected to the transducer array elements L31, and applies electric driving signals to the transducer array elements L31, respectively, thereby achieving ultrasonic emission.

Further, the impedance detection unit L52 contains N impedance detection circuits, the N impedance detection circuits are electrically connected to the metal conductors L221 of the skin impedance detection electrode array, detection resistors R are connected at signal return ends of the metal conductors L221, and the other ends of the detection resistors R are connected to the ground. In this embodiment, N=8, the resistance of the detection resistors R is 50KΩ.

In this embodiment, the electric excitation signal is an alternating current signal, and this embodiment can detect the resistive, capacitive, and inductive values of the skin tissue. The alternating current signal can be set to different frequencies to enable the detection of layered impedance in the depth direction of the skin tissue.

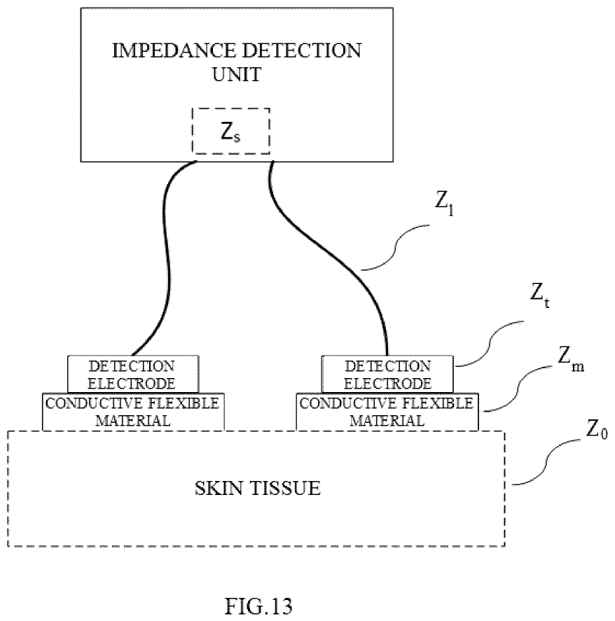

In conjunction of FIG. 13, in this embodiment, the equivalent impedance Zₜ of the detection electrodes, the equivalent impedance Z of the metal conductors L221, and the equivalent impedance Zₛ of the signal link on the impedance detection unit are at a milliohm level, and can be ignored; the equivalent impedance Zₘ of the conductive flexible material L11 is 1 KΩ.

In another embodiment, the electric excitation signal is a direct current signal, and this embodiment can only detect the resistive value of the skin tissue and cannot detect the inductive and capacitive values of the skin tissue, but the impedance detection unit is simple in design and low in cost, suitable for the cases with low detection requirements.

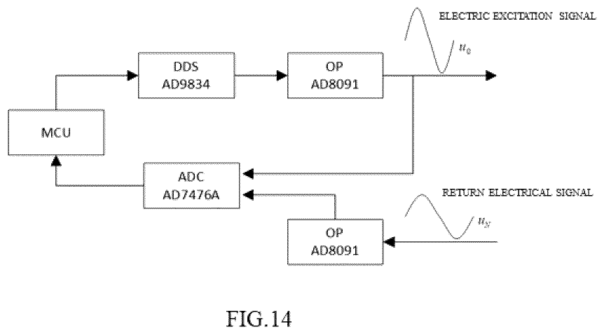

In conjunction of FIG. 14, in this embodiment, the control unit is an MCU, which can be a common control chip like ARM and FPGA. The impedance detection circuit shown contains a DDS chip AD9834, an operational amplifier OP chip AD8091 and an ADC chip AD7476A. The MCU controls the DDS chip AD9834 to generate an electrical signal, which generates the electric excitation signal after amplification by the operational amplifier AD8091. Further, the return electrical signal is transmitted to the ADC chip AD7476A after amplification by the operational amplifier AD8091, and the MCU can detect the return electrical signal by means of the ADC chip AD7476A.

In this embodiment, the control method of the driving device comprises the following steps:
S01: controlling the impedance detection unit to apply an electric excitation signal u₀ to each channel impedance detection electrode in turn, wherein the electric excitation signal u₀ is an alternating current signal or a direct current signal, if the electric excitation signal u₀ is an alternating current signal, then going to S02 and S03, and if the electric excitation signal u₀ is a direct current signal, then going to S02 and S04;
S02: controlling the impedance detection unit to detect a return electrical signal uₙ of each channel impedance detection electrode after passing through the skin tissue;
S03: extracting three periodic signals u₀′ after the electric excitation signal u₀ passes the zero point for the first time, and three periodic signals uₙ′ after the return electrical signal uₙ passes the zero point for the first time if the electric excitation signal u₀ is an alternating current signal; at the same time, calculating the complete impedance Z_{total,n} of the entire signal link for each channel according to the following formula:

$${Z}_{total , n} = \frac{{u}_{0}^{′} R}{{u}_{n}^{′}} - R ;$$

where, u₀ is one or more periodic signals after the electric excitation signal u₀ passes the zero point for the first time, uₙ′ is one or more periodic signals after the return electrical signal uₙ passes the zero point for the first time, and R is the resistance of the detection resistor.
S04: calculating the complete impedance Z_{total,n} of the entire signal link for each channel according to the following formula by the control unit if the electric excitation signal u₀ is a direct current signal:

$${Z}_{total , n} = \frac{{u}_{0} R}{{u}_{n}} - R ;$$

where, u₀ is the electric excitation signal, uₙ is the return electrical signal, and R is the resistance of a detection resistor.
S05: calculating the impedance Zₙ of the skin tissue according to the following formula:

$${Z}_{n} = {Z}_{total , n} - 2 {Z}_{t , n} - 2 {Z}_{m , n} - 2 {Z}_{l , n} - {Z}_{s , n}$$

where, n is a channel number, Z_{t,n} is the equivalent impedance of the detection electrode L21, Z_{m,n} is the equivalent impedance of the conductive flexible material, Z_{l,n} is the equivalent impedance of the metal conductors, and Z_{s,n} is the equivalent impedance of the signal link on the impedance detection unit;
S06: calculating the deviation Zₛₙ of each value Zₙ;

$${Z}_{sn} = \frac{{Z}_{n} - μ}{\sqrt{\frac{{\sum }_{n = 1}^{N} {( {Z}_{n} - μ )}^{2}}{N}}}$$

Where, μ is a mean value of all values Zₙ;
S07: comparing Zₛₙ with a preset deviation threshold, and marking channels that exceed the deviation threshold as abnormal channels;
S08: applying an electrical signal to non-abnormal channels by means of the array element driving unit, thereby achieving ultrasonic emission;
S09: during ultrasonic emission, detecting the skin impedance values of N channels in turn by means of the impedance detection unit again, and marking them as Zₙ′;
S10: calculating the change in skin impedance of each channel after ultrasonic irradiation according to the following formula and marking it as Δkₙ:

$$Δ {k}_{n} = \vert 1 - \frac{{{Z}_{n}}^{′} - {Z}_{n}}{{Z}_{n}} \vert$$

S11: comparing Δkₙ with a preset dose threshold, stopping emitting ultrasonic waves to channels of which Δkₙ is greater than the dose threshold, and continuing to emit ultrasonic waves to other channels; and

S12: repeating S03 to S07 until all channels reach the dose threshold.

The basic principle, main features and advantages of the present invention are shown and described above. Those of ordinary skill in the art should understand that the above embodiments do not limit the scope of protection of the present invention in any form and that all technical schemes obtained by means of equivalent replacement, etc. fall within the scope of protection of the present invention. The parts not covered by the present invention are identical to or can be implemented using the prior art.

1. A flexible ultrasonic transducer capable of detecting skin impedance, wherein the ultrasonic transducer comprises a coupling layer, an electrode layer, an array element layer and a backing layer,
the coupling layer comprises a non-conductive flexible material and a conductive flexible material;
the electrode layer is located below the coupling layer, and a second metal layer for connecting the conductive flexible material and a driving device, and a fourth metal layer for connecting the ground are arranged in the electrode layer;
the array element layer is fit together with the electrode layer and comprises N transducer array elements, array element drive wires and a flexible material, and the flexible material fills among the transducer array elements;
the backing layer is located below the array element layer.

2. The flexible ultrasonic transducer capable of detecting skin impedance according to claim 1, wherein the coupling layer is an odd number of times as thick as a quarter of the ultrasonic wavelength.

3. The flexible ultrasonic transducer capable of detecting skin impedance according to claim 1, wherein an orifice is opened in the non-conductive flexible material and filled with the conductive flexible material.

4. The flexible ultrasonic transducer capable of detecting skin impedance according to claim 1, wherein the direct current impedance and alternating current impedance of the non-conductive flexible material are greater than the measured skin impedance; the direct current impedance and alternating current impedance of the conductive flexible material are smaller than the measured skin impedance.

5. The flexible ultrasonic transducer capable of detecting skin impedance according to claim 2, wherein cotton slots are arranged on the coupling layer and intended to contain medicine guide cotton.

6. The flexible ultrasonic transducer capable of detecting skin impedance according to claim 1, wherein the electrode layer comprises a first insulating layer, a second metal layer, a third insulating layer, a fourth metal layer and a fifth insulating layer arranged in a stacking manner in turn.

7. The flexible ultrasonic transducer capable of detecting skin impedance according to claim 6, wherein the second metal layer is provided with a skin impedance detection electrode array, the skin impedance detection electrode array comprises N metal conductors, and two ends of each metal conductor are provided with a first metal electrode and a second metal electrode, respectively.

8. The flexible ultrasonic transducer capable of detecting skin impedance according to claim 7, wherein the metal conductors adopt a serpentine structure that curves back and forth.

9. The flexible ultrasonic transducer capable of detecting skin impedance according to claim 7, wherein the first metal electrode is electrically connected to the conductive flexible material, and the second metal electrode is electrically connected to the driving device.

10. The flexible ultrasonic transducer capable of detecting skin impedance according to claim 7, wherein an orifice is arranged in the first insulating layer above the first metal electrode and filled with the conductive flexible material, and the first metal electrode is electrically connected to the conductive flexible material.

11. The flexible ultrasonic transducer capable of detecting skin impedance according to claim 6, wherein the fourth metal layer (L24) is electrically connected to the ground.

12. The flexible ultrasonic transducer capable of detecting skin impedance according to claim 1, wherein the array element layer comprises N transducer array elements, array element drive wires and a flexible material, and the flexible material fills among the transducer array elements, and the array element drive wires are arranged below the transducer array elements and the flexible material and electrically connected to the transducer array elements.

13. The flexible ultrasonic transducer capable of detecting skin impedance according to claim 12, wherein the minimum distance Dₘᵢₙ between the transducer array elements is calculated according to the following formula:
$${D}_{min} = 2 h sin \frac{π - β}{2}$$
where, h is the thickness of the transducer array elements, and β is a conformal skin angle required for the ultrasonic transducer.

14. The flexible ultrasonic transducer capable of detecting skin impedance according to claim 12, wherein top electrodes of the transducer array elements extend through the side faces of the array elements to the bottom surfaces of the array elements and are connected to the array element drive wires, and bottom electrodes of the transducer array elements are connected to the array element drive wires.

15. The flexible ultrasonic transducer capable of detecting skin impedance according to claim 12, wherein the transducer array elements are planar array elements or curved array elements, the convex sides of the curved array elements face upward, the thickness of the non-conductive flexible material above the curved array elements is greater than D_{L03}, and the calculation formula for D_{L03} is as follows:
$${D}_{L 03} = \frac{D + d}{2 tan α} - r - h$$
where, α is the radiation angle of the curved array elements, r is the curvature radius of the curved array elements, d is the width of the curved array elements, h is the thickness of the curved array elements, and D is the actual distance between array elements.

16. A driving device used in the flexible ultrasonic transducer in claim 1, wherein the driving device comprises a control unit, an impedance detection unit and an array element driving unit, the control unit is electrically connected to the impedance detection unit and the array element driving unit, and the array element driving unit is electrically connected to the transducer array elements, and applies electric driving signals to the transducer array elements, respectively, thereby achieving ultrasonic emission.

17. The driving device according to claim 16, wherein the impedance detection unit contains N impedance detection circuits, the N impedance detection circuits are electrically connected to the metal conductors of the skin impedance detection electrode array and detection resistors R are connected at signal return ends of the metal conductors; the impedance detection unit controls each impedance detection circuit in turn to apply an electrical excitation signal u₀ to the metal conductors, and at the same time, detects the return electrical signal uₙ of each channel after passing through the skin tissue, where n is the channel number.

18. The driving device according to claim 17, wherein the electric excitation signal u₀ is an alternating current signal, the control unit extracts one or more periodic signals u₀′ after the electric excitation signal u₀ passes the zero point for the first time, and one or more periodic signals uₙ′ after the return electrical signal uₙ passes the zero point for the first time, and the periodic signals u₀′ and uₙ′ have the same number of periods.

19. The driving device according to claim 18, wherein the control unit calculates the complete impedance Z_{total,n} of the entire signal link for each channel according to the following formula:
$${Z}_{total , n} = \frac{{{u}_{0}}^{′} R}{{{u}_{n}}^{′}} - R$$
where, u₀′ is one or more periodic signals after the electric excitation signal u₀ passes the zero point for the first time, uₙ′ is one or more periodic signals after the return electrical signal uₙ passes the zero point for the first time, and R is the resistance of a detection resistor;
the control unit calculates the impedance Zₙ of the skin tissue according to the following formula:
$${Z}_{n} = {Z}_{total , n} - 2 {Z}_{t , n} - 2 {Z}_{m , n} - 2 {Z}_{l , n} - {Z}_{s , n}$$
where, n is a channel number, Z_{t,n} is the equivalent impedance of the detection electrode L21, Z_{m,n} is the equivalent impedance of the conductive flexible material, Z_{l,n} is the equivalent impedance of the metal conductors, and Z_{s,n} is the equivalent impedance of the signal link on the impedance detection unit.

20. The driving device according to claim 17, wherein the electric excitation signal u₀ is a direct current signal, and the control unit calculates the complete impedance Z_{total,n} of the entire signal link for each channel according to the following formula:
$${Z}_{total , n} = \frac{{u}_{0} R}{{u}_{n}} - R$$
where, u₀ is an electric excitation signal, uₙ is a return electrical signal, and R is the resistance of a detection resistor;
the control unit calculates the impedance Zₙ of the skin tissue according to the following formula:
$${Z}_{n} = {Z}_{total , n} - 2 {Z}_{t , n} - 2 {Z}_{m , n} - 2 {Z}_{l , n} - {Z}_{s , n}$$
where, n is a channel number, Z_{l,n} is the equivalent impedance of the detection electrode L21, Z_{m,n} is the equivalent impedance of the conductive flexible material, Z_{l,n} is the equivalent impedance of the metal conductors, and Z_{s,n} is the equivalent impedance of the signal link on the impedance detection unit.

21. A control method of the driving device according to claim 16, wherein the method comprises the following steps:
S01: controlling the impedance detection unit to apply an electric excitation signal u₀ to each channel impedance detection electrode in turn, wherein the electric excitation signal u₀ is an alternating current signal or a direct current signal, if the electric excitation signal u₀ is an alternating current signal, then going to S02 and S03, and if the electric excitation signal u₀ is a direct current signal, then going to S02 and S04;
S02: controlling the impedance detection unit to detect a return electrical signal uₙ of each channel impedance detection electrode after passing through the skin tissue;
S03: extracting three periodic signals u₀′ after the electric excitation signal u₀ passes the zero point for the first time, and three periodic signals uₙ′ after the return electrical signal uₙ passes the zero point for the first time if the electric excitation signal is an alternating current signal u₀; at the same time, calculating the complete impedance Z_{total,n} of the entire signal link for each channel according to the following formula:
$${Z}_{total . n} = \frac{{{u}_{0}}^{′} R}{{{u}_{n}}^{′}} - R$$
where, u₀′ is one or more periodic signals after the electric excitation signal u₀ passes the zero point for the first time, uₙ′ is one or more periodic signals after the return electrical signal uₙ passes the zero point for the first time, and R is the resistance of a detection resistor;
S04: calculating the complete impedance Z_{total,n} of the entire signal link for each channel according to the following formula by the control unit if the electric excitation signal u₀ is a direct current signal:
$${Z}_{total , n} = \frac{{u}_{0} R}{{u}_{n}} - R ;$$
where, u₀ is an electric excitation signal, uₙ is a return electrical signal, and R is the resistance of a detection resistor;
S05: calculating the impedance Zₙ of the skin tissue according to the following formula:
$${Z}_{n} = {Z}_{total , n} - 2 {Z}_{t , n} - 2 {Z}_{m , n} - 2 {Z}_{l , n} - {Z}_{s , n}$$
where, n is a channel number, Zen is the equivalent impedance of the detection electrode L21, Z_{m,n} is the equivalent impedance of the conductive flexible material, Z_{l,n} is the equivalent impedance of the metal conductors, and Z_{s,n} is the equivalent impedance of the signal link on the impedance detection unit;
S06: calculating the deviation Zₛₙ of each value Zₙ;
$${Z}_{sn} = \frac{{Z}_{n} - μ}{\sqrt{\frac{{\sum }_{n = 1}^{N} {( {Z}_{n} - μ )}^{2}}{N}}}$$
where, μ is a mean value of all values Zₙ;
S07: comparing Zₛₙ with a preset deviation threshold, and marking channels that exceed the deviation threshold as abnormal channels;
S08: applying an electrical signal to non-abnormal channels by means of the array element driving unit, thereby achieving ultrasonic emission;
S09: during ultrasonic emission, detecting the skin impedance values of N channels in turn by means of the impedance detection unit again, and marking them as Zₙ′;
S10: calculating the change in skin impedance of each channel after ultrasonic irradiation according to the following formula and marking it as Δkₙ:
$$Δ {k}_{n} = \vert 1 - \frac{{{Z}_{n}}^{′} - {Z}_{n}}{{Z}_{n}} \vert$$
S11: comparing Δkₙ with a preset dose threshold, stopping emitting ultrasonic waves to channels of which Δkₙ is greater than the dose threshold, and continuing to emit ultrasonic waves to other channels; and
S12: repeating S03 to S07 until all channels reach the dose threshold.
